(19) United States
(12) Plant Patent Application Publication
(10) Pub. No.: US 2002/0133863 P1
(43) Pub. Date: Sep. 19, 2002

(54) MISCANTHUS SINENSIS PLANT NAMED 'LITTLE ZEBRA'

(76) Inventor: Thomas M. Walsh, Nunica, MI (US)

Correspondence Address: David S. MacKenzie, Hortech, Inc., P.O. Box 533, Spring Lake, MI, 49456, US

(21) Appl. No.: 09/765,699

(22) Filed: Mar. 19, 2001

(51) Int. Cl. A01H 5/00

(52) U.S. Cl. PLT/384

(57) ABSTRACT

A Japanese silver grass named ‘Little Zebra’, characterized by its narrow, green and yellow banded foliage, dwarf habit, and silvery white late-summerborne flowers.

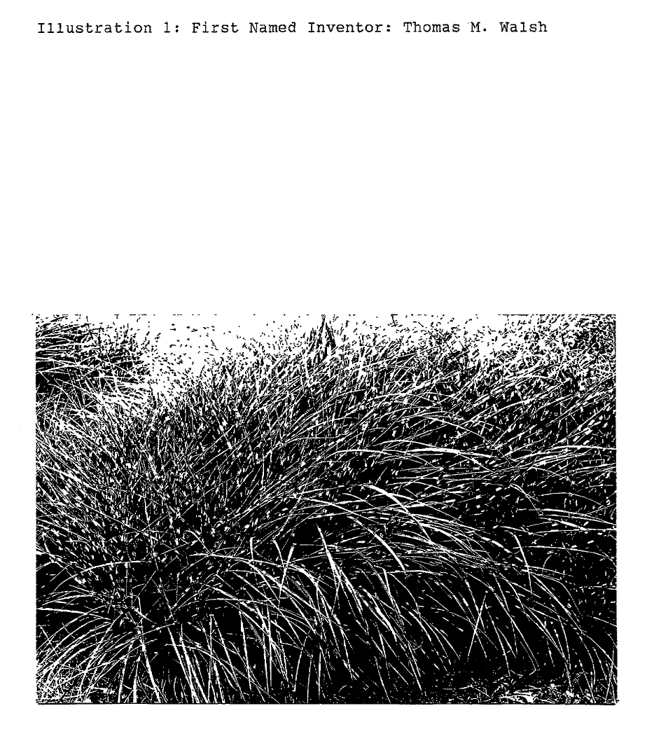

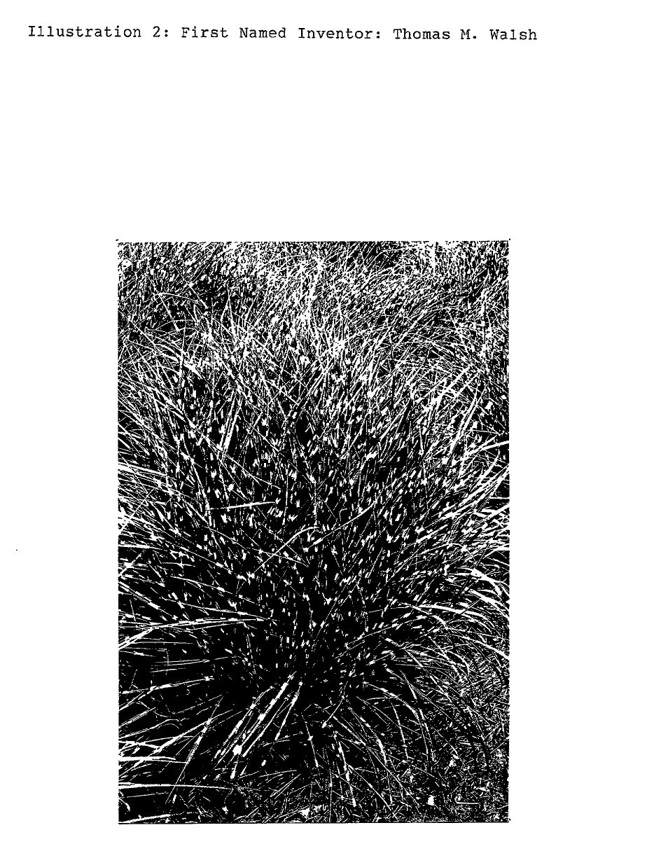

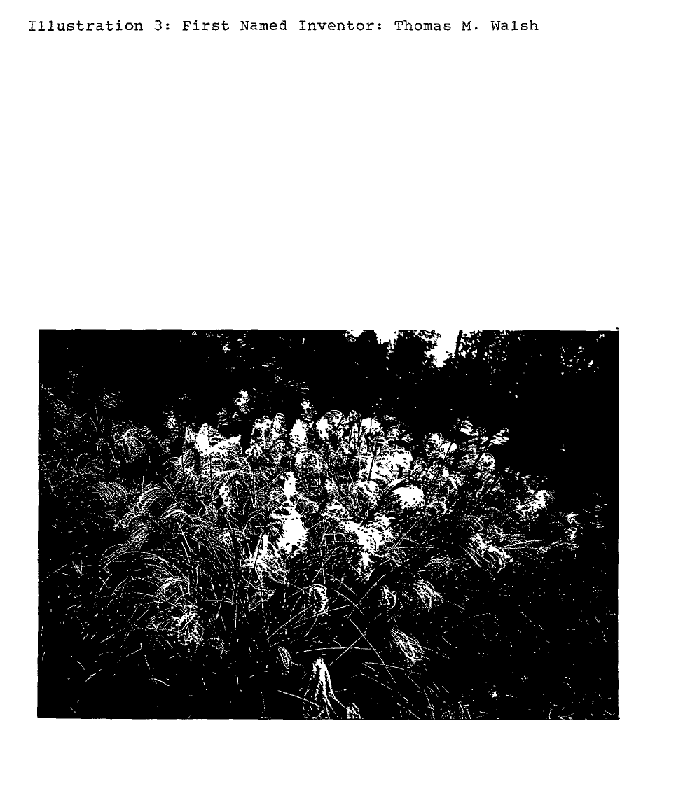

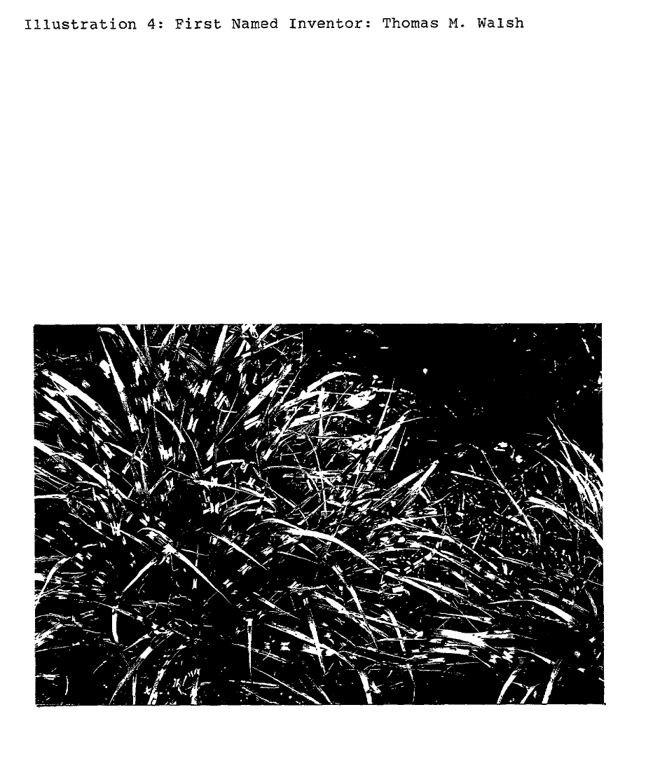

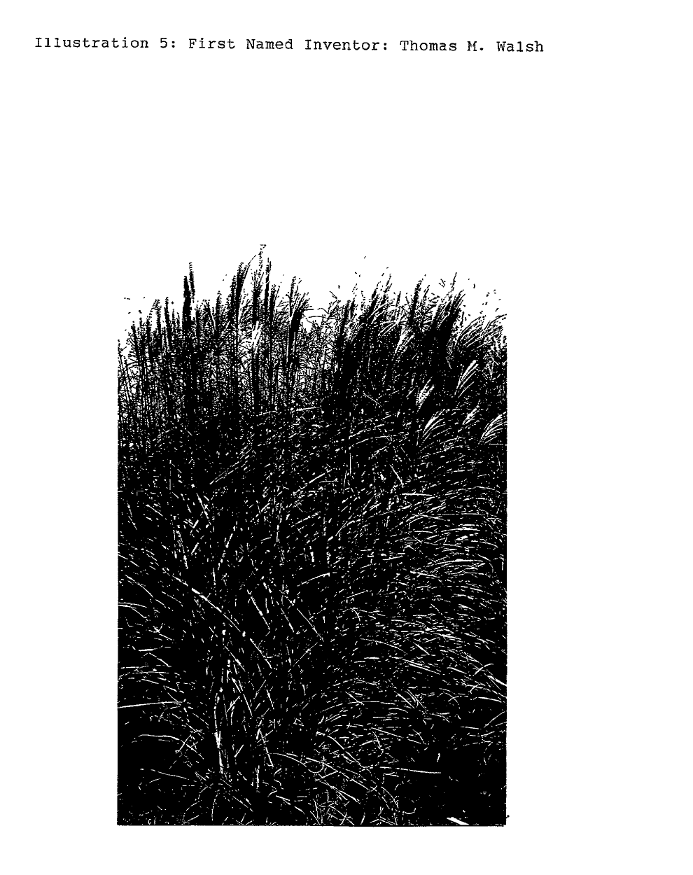

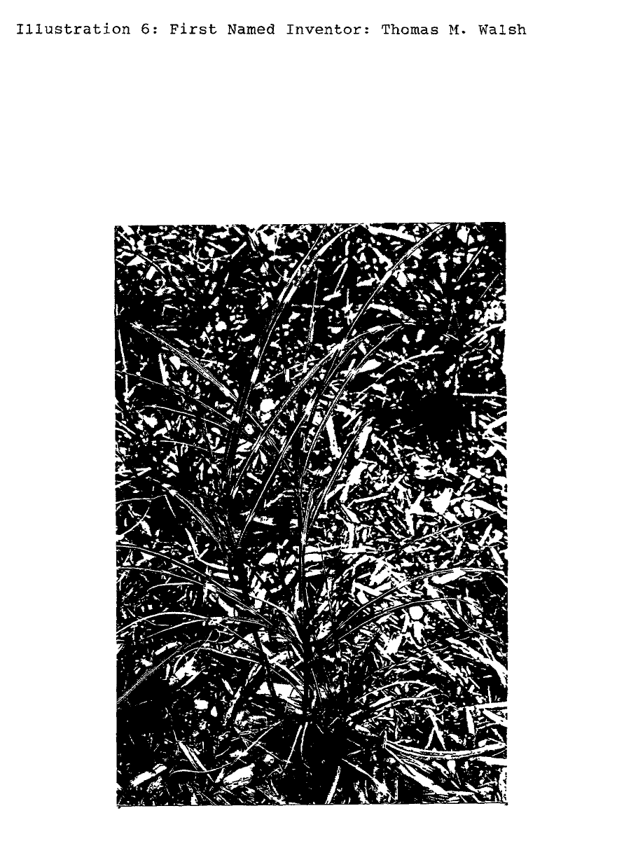

### CROSS REFERENCE

This application does not cross reference with other related applications.

### FEDERALLY SPONSORED RESEARCH

This application does not relate to any federally sponsored research.

### BACKGROUND OF INVENTION

‘Little Zebra’ originated as a bud sport (mustation) from the species Miscanthus sinensis and is similar to the parent species only in the habit (shape) of the plant. The first successful asexual reproduction of ‘Little Zebra’ was carried out by its discoverer, Thomas M. Walsh. This propagation took place under controlled conditions in Nunica, Mich., on Aug. 1, 1995.

The mutated bud was divided (with a section of crown, foliage, and roots from the parent plant) and potted in an organic growing medium. This division survived and was transplanted to an outdoor growing bed where it was grown to mature size.

### SUMMARY OF INVENTION

Once rooted, the variegated sport was planted out in the landscape where it displayed narrow green and yellow striped leaves, dwarf habit (in relation to the parent species), and late summertime bloom time.

### DESCRIPTION OF THE VARIOUS VIEWS OF THE DRAWINGS

The accompanying photographic illustrations show typical foliage and growth habit of the cultivar and the closest known comparison plants (Miscanthus sinensis ‘Hinjo’ (a.k.a. ‘Little Nicky’), Miscanthus sinensis ‘Puntchen’, and Miscanthus sinensis ‘Zwergzebra’ with color being as accurate as possible with renditions of this type. The photographs were taken at 11 A.M. on Sep. 10, 2000 under cloudy conditions, in Nunica, Mich. The film used was Kodak Gold ISO 200 exposed for 1/30 second at F 5.6 using no filters.

Illustrations 1-3 are perspective views of Miscanthus sinensis ‘Little Zebra’. Illustration 4 is a perspective view of Miscanthus sinensis ‘Hinjo’. Illustration 5 is a perspective view of Miscanthus sinensis ‘Puenktchen’. Illustration 6 is a perspective view of Miscanthus sinensis ‘Zwergzebra’.

### DETAILED BOTANICAL DESCRIPTION

‘Little Zebra’ has not been observed under all possible environmental conditions, and the phenotype may vary significantly with variations in environment; such as temperature light intensity and day length. The following observations, measurements, and comparisons describe this plant as grown in Nunica, Mich. when grown outdoors without cover.

The following traits have been repeatedly observed and are determined to be the basic characteristics of ‘Little Zebra, which in combination distinguish this Japanese silver grass as a new and distinct cultivar. The color codes correspond to The Royal Horticultural Society Color Chart.

1. Deciduous foliage is linear, alternate, crowded at the base of the plant. Each is attached to a cylindrical, pith-filled, jointed culm (stem), ⅛ to 3/16 inch wide, colored green 134-A. The culms in the center of each clump are held erect. Those farther out tend to arch away from the center of the plant, on as much as a 15 degree angle. The distance between the leaves ranges from ¼ inch at the base of the culm increasing to 5 inches toward the end of the culm.

2. Flattened mostly basal leaf blades are attached to the culm at their ligules which are colored green 134-A. Each ligule is hairless and encircles the culm ½ way around. Leaf blades range from 10 to 13 inches long, 3/16 to ¼ inch wide, and are finely bristle haired along their edges, except for about 1 inch at the base of the blade which is smooth edged. Leaf blades gradually taper to a slender pointed tip and are oriented on an acute angle of approximately 30 degrees in relation to the distal end of the culm.

3. Each leaf blade is colored with alternating horizontal bands of yellow and green which extend from one edge of the leaf to the other; the green bands, green 134-A, range from 1 to 3 inches in height. The yellow bands, yellow 8-C, range from ⅜ to 1 inch in height.

4. Each leaf blade has a central prominent longitudinal midrib, ⅛ inch wide, color green 134-D. The overall veination pattern is longitudinal/parallel with the thin (less than 1/32 inch wide) veins being relatively unnoticable, and colored the same as the corresponding surrounding leaf tissue.

5. Below each ligule is a sheath that ranges from ¼ inch in length, at the base of the culm, to 5 inches in length, at the very top of the culm. Each leaf sheath originates at a slightly swollen node, colored green 134-B, and ranging from 3/16 to ¼ inch wide. The sheaths fully encircle the culm from the node to about ⅓ way up to the ligule, the top ⅔ of the sheath encircles about ½ way round the culm.

6. Each culm ultimately terminates in a late summerborne corymbose panicle (inflorescence) of 10 to 13 racemes; each raceme to 6 inches long, axis of the panicles shorter than or about as long as the racemes. The tiny bisexual flowers (spikelets) are grayed purple in youth, 184-C, maturing to grayed yellow 161-D.

7. Flowers are paired, ⅛ inch long, with protruding silky silvery white, 155-D, hairlike unbranched stigmas. The 1/16 inch long cigar-shaped anthers are carried upon hair-thin filaments and bear yellow-orange, 14-B, pollen. The flowers are not fragrant.

8. No seed formation has been observed in Michigan but farther south, where the growing season is longer, one would expect to see tiny grain formed.

9. The general growth habit of ‘Little Zebra’ is dense-clump forming.

Clumps have been observed to measure 1½ feet across at ground level after four years. The mode of expansion is short rhizomes.

Other cultivars of Miscanthus sinensis include ‘Hinjo’ which reaches 4 feet tall, blooms sparsely, and differs primarily in having much wider leaves which are frequently discolored with red blotches. ‘Puenktchen’, which differs in being 5 to 7 feet tall, wider leaved, and its green and yellow banded leaves change to all-green during the course of the growing season. And, ‘Zwergzebra’, which differs in being 5 to 6 feet tall, with broader foliage, comparatively few yellow bands, and culm bases colored reddish purple.

1. The present invention comprises a new and distinct cultivar of Miscanthus sinensis, hereafter referred to by the cultivar name ‘Little Zebra’
‘Little Zebra’ is a new and distinct variety of Japanese silver grass, a cold hardy deciduous perennial clump forming ornamental grass.
‘Little Zebra’ is unique and outstanding for its dwarf habit in combination with distinct sunfast yellow and green banded, narrow bladed foliage.
